(19) United States
(12) Patent Application Publication
(10) Pub. No.: US 2021/0059247 A1
(43) Pub. Date: Mar. 4, 2021

(54) PREPARATION OF LONG-LASTING ANTIBACTERIAL CORE-SHELL AGENT AND APPLICATION FOR THE SOLID MEDIA OR SURFACE COATING

(71) Applicant: MEGANITE, INC., ONTARIO, CA (US)

(72) Inventors: HUANG CHIH LU, TAICHUNG CITY (TW); CHUN AN LAI, LAGUNA NIGUEL, CA (US)

(73) Assignee: MEGANITE, INC., ONTARIO, CA (US)

(21) Appl. No.: 17/006,864

(22) Filed: Aug. 30, 2020

Related U.S. Application Data
(60) Provisional application No. 62/894,760, filed on Aug. 31, 2019.

(51) Int. Cl. A01N 25/26; A01N 59/20

(52) U.S. Cl. CPC A01N 25/26; B82Y 5/00; A01N 59/20

(57) ABSTRACT

A long-lasting antibacterial core-shell agent, a method for preparing a long-lasting antibacterial agent and method for preparing a solid media utilizing a long-lasting antibacterial core-shell agent. The long-lasting antibacterial core-shell agent comprises an antibacterial core composed of an antibacterial metal powder, a poorly soluble metal salt and a metal oxide; and an outer shell composed of a porous oxide. The solid media prepared utilizing the long-lasting antibacterial core-shell agent has the ability to resist ultraviolet damage, inhibits oxygen oxidation, slowly releases metal ions and thereby exhibits long-term release of antibacterial ions and longitudinal antibacterial uniformity.

This application claims the benefit of U.S. Provisional Application No. 62/894,760 filed on Aug. 31, 2019.

### BACKGROUND OF THE DISCLOSURE

### Technical Field of the Disclosure

The present invention relates generally to the treatment of multi-functional materials for interior and exterior design uses, and more specifically to a method of preparing a long-lasting antibacterial core-shell agent and its application for the solid media or surface coating.

### Description of the Related Art

Core-shell based long-term antibacterial characteristic particles can be used for solid media or surface coating. The different structures of core and shell materials can also be used to control antibacterial ability. Generally, normal antibacterial products can be used at home and stronger antibacterial products can be used in hospitals.

Antibacterial particles can be applied for acrylic, epoxy, unsaturated polymer based resin which can be constructed on solid media or surface coating. Many commercially available antibacterial solid media treatments can release antibacterial components on the surface of materials for only 24 hours and then the effect is weakened and needs to be restored.

In some other core-shell based antibacterial agents, the addition of resin made it difficult to release key components of antibacterial materials. Inorganic types of antibacterial matters were previously formulated in a powder-mixing manner to prepare an antibacterial agent formula that would conform to the characteristics of the product. But such long-lasting antibacterial agents were not easy to control under normal conditions.

Some prior treatments included covering antibacterial components to the outside of a metal oxide or metal hydroxide substrate. In some cases, solid surfaces would be pre-treated or restored for maintaining the antibacterial characteristics. However, these processes were not easy to commercialize, and commercial products were not easy to be pre-treated or restored by end users since the process would require some form of cutting, fabrication, and installation. Such existing antibacterial materials also fails to resist ultraviolet damage and fails to provide antibacterial uniformity.

Thus, there is a need for a long-lasting antibacterial core-shell agent, a method for preparing the long-lasting antibacterial agent and a method for preparing a solid media utilizing the long-lasting antibacterial core-shell agent. Such a needed inorganic antibacterial core-shell agent would allow preparation of a long-lasting antibacterial solid media or surface coating. Such an antibacterial agent and the solid media would resist ultraviolet damage, inhibits oxygen oxidation, and slowly release metal ions. Such a needed structure would exhibit long-term release of antibacterial ions and longitudinal antibacterial uniformity. Such an antibacterial agent and the solid media would maintain a consistent antibacterial ability after various surface grinding and treating processes. Such a structure has a unique ratio of acrylic resin that allows continuous release of the antibacterial component. Such an antibacterial agent and the solid media do not need to be restored or retreated to continue antibacterial activity and the antibacterial ability of the material is elevated for more years. Such a needed solid media with antibacterial characteristic enable ease of use for the users and enhance hygienic property. Such a needed solid media allows fabrication into multi-layer structure and can be used horizontally and vertically for interior and exterior designs. The present embodiment overcomes shortcomings in the field by accomplishing these critical objectives.

### SUMMARY OF THE DISCLOSURE

To minimize the limitations found in the existing systems and methods, and to minimize other limitations that will be apparent upon the reading of the specifications, a preferred embodiment of the present invention provides a long-lasting antibacterial core-shell agent, a method for preparing the long-lasting antibacterial agent and method for preparing a solid media utilizing the long-lasting antibacterial core-shell agent.

The long-lasting antibacterial core-shell agent comprises an antibacterial core composed of an antibacterial metal powder, a poorly soluble metal salt and a metal oxide; and an outer shell composed of a porous oxide. The antibacterial metal powder is nano-sized ranging range from 30 nm to 3000 nm and is made from metals selected from the group consisting of: Mercury (Hg), Silver (Ag), Arsenic (As), Copper (Cu), Zinc (Zn), Cobalt (Co), Nickel (Ni), Aluminum (Al), Titanium (Ti) and Manganese (Mn). The poorly soluble metal salt is selected from the group consisting of: Silver Chloride (AgCl), Copper Chloride (CuCl) and Mercury Chloride (HgCl₂). The metal oxide is selected from the group consisting of: Copper Oxide (CuO) and Zinc Oxide (ZnO). The outer shell of porous oxide is selected from the group consisting of: silicon dioxide (SiO₂), titanium dioxide (TiO₂), aluminum oxide (Al₂O₃) and aluminum hydroxide (Al(OH)₃).

The method for preparing a long-lasting antibacterial agent comprises the steps of synthesizing an anti-bacterial core having an antibacterial metal powder, a poorly soluble metal salt and a metal oxide and forming a porous oxide shell over the anti-bacterial core by fluidizing and agitating an organic polymer. The synthesis of the anti-bacterial core comprises the steps of preparing a surfactant solution of a cationic surfactant and an organic solvent with a concentration of 0.01-0.5 M. Then adding a certain amount as of aqueous metal nitrate solution with a concentration of 0.05-0.1 M to the surfactant solution by fixing the mole ratio of water to surfactant as 4 to 6, to form a water/oil micro-emulsion. Stirring the water/oil micro-emulsion at a constant speed at room temperature and then adding a reducing agent drop-wise to reduce metal ions to metal. Then preparing nano particles of metal oxide by adjusting the hydrolysis ratio and adding a protective agent and preparing poorly soluble metal salt from at least two soluble metal salts by mechanochemical processing and precipitation reaction synthesis. In this embodiment, permanently charged quaternary ammonium cations are used as surfactants which include alkyltrimethylammonium salts like cetyl trimethylammonium bromide (CTAB) and cetyl trimethylammonium chloride (CTAC). The organic solvent includes alcohols selected from the group consisting of: ethanol and isopropyl alcohol. The reducing agent used in this embodiment includes antioxidant molecules selected from the group consisting of: sodium borohydride and ascorbic acid. The protective agents used for preparing metal oxides are surfactants and polymers. In this embodiment, dimethylaminoborane and poly (acrylic acid, sodium salt) is used.

The metal oxide is made from precursors selected from the group consisting of: metal nitrate, metal chloride, metal acetate and metal sulfate. The organic polymer for making the porous oxide is selected from the group consisting of: alucone, silicon alkoxide (silicone), zinc alkoxide (zincone), titanium alkoxide (titanicone), and zirconium alkoxide (zircone).

The method for preparing the solid media utilizing the long-lasting antibacterial core-shell agent comprises the steps of mixing well aluminum hydroxide (Al(OH)) powder, long lasting anti-bacterial core-shell agent and stone fragments using vibratory compaction in a vacuum environment to form a mixture. Then adding unsaturated polymer resins to the mixture, under controlled compaction pressure, vibration frequency and vacuum condition and obtaining the solid media with high compressive strength, low water absorption, suitable density and flexural strength. The percentage of each component of the solid media includes 60-69% of Al(OH)₃ powder, 1-10% of the long lasting anti-bacterial core-shell agent, 10% of stone fragments and 8% of unsaturated polymer resins.

A first objective of the present embodiment is to provide a long-lasting antibacterial core-shell agent, a method for preparing the long-lasting antibacterial agent and a method for preparing a solid media utilizing the long-lasting antibacterial core-shell agent.

A second objective of the present embodiment is to provide an inorganic antibacterial core-shell agent that allows preparation of a long-lasting antibacterial solid media or surface coating.

A third objective of the present embodiment is to provide an antibacterial agent and a solid media that resists ultraviolet damage, inhibits oxygen oxidation, and slowly release metal ions.

A fourth objective of the present embodiment is to provide an improved structure that exhibits long-term release of antibacterial ions and longitudinal antibacterial uniformity.

A fifth objective of the present embodiment is to provide an antibacterial agent and a solid media that maintains consistent antibacterial ability after various surface grinding and treating processes.

Another objective of the present embodiment is to provide a structure having unique ratio of acrylic resin that allows continuous release of the antibacterial component.

Yet another objective of the present embodiment is to provide an antibacterial agent and a solid media that does not need to be restored or retreated to continue antibacterial activity and the antibacterial ability of the material is elevated for more years.

Still another objective of the present embodiment is to provide a solid media with antibacterial characteristics that enable ease of use for the users and enhance hygienic property.

Further another objective of the present embodiment is to provide a solid media that allows fabrication into multi-layer structure and which can be used horizontally and vertically for interior and exterior designs.

These and other advantages and features of the present invention are described with specificity so as to make the present invention understandable to one of ordinary skill in the art.

### BRIEF DESCRIPTION OF THE DRAWINGS

In order to enhance their clarity and improve the understanding of the various elements and embodiment, elements in the figures have not necessarily been drawn to scale. Furthermore, elements that are known to be common and well understood to those in the industry are not depicted in order to provide a clear view of the various embodiments of the invention. Thus, the drawings are generalized in form in the interest of clarity and conciseness.

FIGS. 1A-1H illustrate cross-sectional views of different types of currently existing core-shell particles;

FIG. 2 illustrates a cross-sectional view of a long-lasting antibacterial core-shell particle in accordance with the preferred embodiment of the present invention;

FIG. 3 illustrates a method for preparing the long-lasting antibacterial core-shell agent in accordance with the preferred embodiment of the present invention; and

FIG. 4 illustrates a method for preparing a solid media utilizing the long-lasting antibacterial core-shell agent in accordance with the preferred embodiment of the present invention.

### DETAILED DESCRIPTION OF THE DRAWINGS

In the following discussion that addresses a number of embodiments and applications of the present invention, reference is made to the accompanying drawings that form a part hereof, and in which is shown by way of illustration specific embodiments in which the invention may be practiced. It is to be understood that other embodiments may be utilized and changes may be made without departing from the scope of the present invention.

Various inventive features are described below that can each be used independently of one another or in combination with other features. However, any single inventive feature may not address any of the problems discussed above or only address one of the problems discussed above. Further, one or more of the problems discussed above may not be fully addressed by any of the features described below.

As used herein, the singular forms “a”, “an” and “the” include plural referents unless the context clearly dictates otherwise. “And” as used herein is interchangeably used with “or” unless expressly stated otherwise. As used herein, the term ‘about” means+/−5% of the recited parameter. All embodiments of any aspect of the invention can be used in combination, unless the context clearly dictates otherwise.

Unless the context clearly requires otherwise, throughout the description and the claims, the words ‘comprise’, ‘comprising’, and the like are to be construed in an inclusive sense as opposed to an exclusive or exhaustive sense; that is to say, in the sense of “including, but not limited to”. Words using the singular or plural number also include the plural and singular number, respectively. Additionally, the words “herein,” “wherein”, “whereas”, “above,” and “below” and words of similar import, when used in this application, shall refer to this application as a whole and not to any particular portions of the application.

The description of embodiments of the disclosure is not intended to be exhaustive or to limit the disclosure to the precise form disclosed. While the specific embodiments of, and examples for, the disclosure are described herein for illustrative purposes, various equivalent modifications are possible within the scope of the disclosure, as those skilled in the relevant art will recognize.

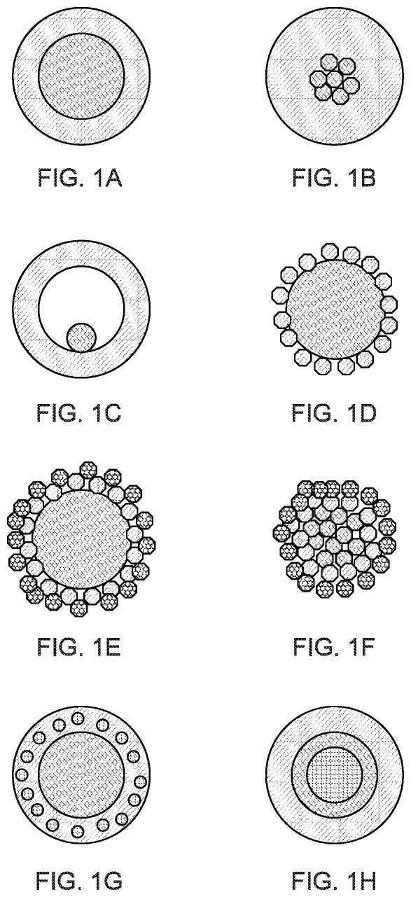

FIGS. 1A-1H illustrate cross-sectional views of different types of currently available core-shell particles. The core and the shell are of different materials with different structures. The core can be a single sphere with attachment of smaller spheres onto a big core sphere (FIGS. 1D and 1E) to form a porous protection shell.

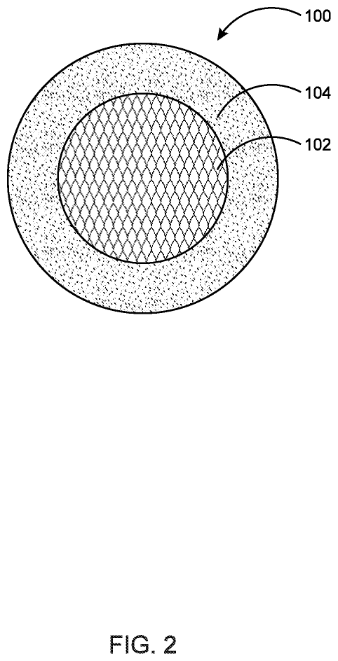

Referring to FIG. 2, a cross-sectional view of a long-lasting antibacterial core-shell particle in accordance with the preferred embodiment of the present invention is illustrated.

Core-shell nanoparticles are an interesting group of nanoparticles as they combine different kinds of materials and therefore are usually multifunctional. They occur in a wide variety based on their size, shape, and structure. The long-lasting antibacterial core-shell particles are a class of particles which contain a core and a shell. The core-shell particle used in the present embodiment of the long lasting anti-bacterial core-shell agent is usually made of the solid single core material and a porous shell. The size of the core particle, the shell thickness and the porosity of the shell are tuned to suit different types of anti-bacterial applications. For these long lasting anti-bacterial agents, they consist of dense metal, metal salt, or metal oxide cores. And these cores can act as metal ion slow-releasing centers with the size range of 30 to 3000 nm.

The long-lasting antibacterial core-shell agent 100 of the present embodiment comprises an antibacterial core 102 and an outer shell 104. The antibacterial core 102 is composed of an antibacterial metal powder, a poorly soluble metal salt and a metal oxide. The outer shell 104 composed of a porous oxide. The antibacterial metal powder of the antibacterial core 102 is nano-sized. The nano-sized metal powder is made from metals selected from the group consisting of: Mercury (Hg), Silver (Ag), Arsenic (As), Copper (Cu), Zinc (Zn), Cobalt (Co), Nickel (Ni), Aluminum (Al), Titanium (Ti) and Manganese (Mn). The size of the particles of the antibacterial metal powder can range from 30 nm to 3000 nm. The poorly soluble metal salt is selected from the group consisting of Silver Chloride (AgCl), Copper Chloride (CuCl) and Mercury Chloride (HgCl₂). The metal oxide of the antibacterial core 102 is selected from the group consisting of: Copper Oxide (CuO) and Zinc Oxide (ZnO). In other embodiments, the long-lasting antibacterial core-shell agent 100 can also employ metal oxides of metals selected from the group consisting of: Mercury (Hg), Silver (Ag), Arsenic (As), Cobalt (Co), Nickel (Ni), Aluminum (Al), Titanium (Ti) and Manganese (Mn).

The porous oxide of the long-lasting antibacterial core-shell agent 100 is selected from the group consisting of: silicon dioxide (SiO₂), titanium dioxide (TiO₂), aluminum oxide (Al₂O₃) and aluminum hydroxide (Ab (OH)₃). The thickness of the particles of porous oxide can range from 10 nm to 1500 nm. The antibacterial core 102 acts as slow-releasing centers of metal ions. The structure of the core-shell 100 has the ability to resist ultraviolet damage, inhibits oxygen oxidation, slowly releases metal ions and thereby exhibits long-term release of antibacterial ions and longitudinal antibacterial uniformity.

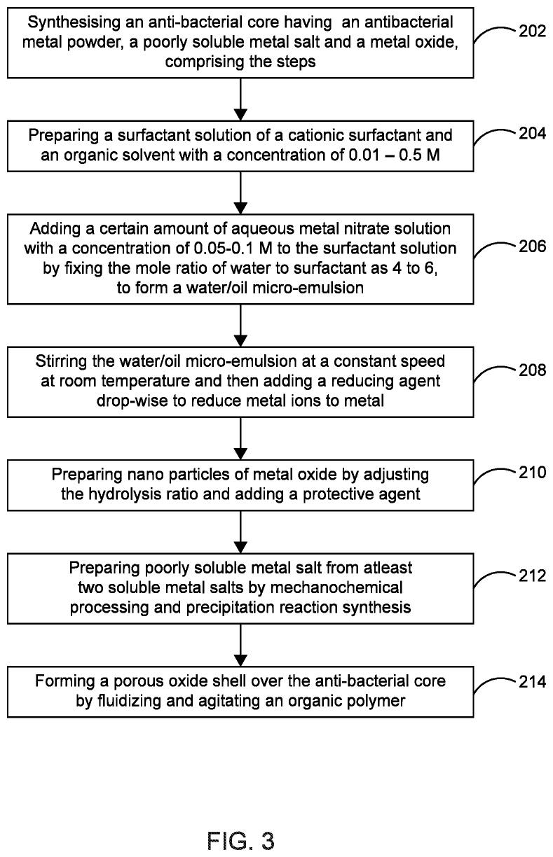

FIG. 3 illustrates a method for preparing the long-lasting antibacterial core-shell agent 100 in accordance with the preferred embodiment of the present invention. For the long-lasting antibacterial core-shell agent 100 of the present embodiment, the core-shell particles are synthesized by a two-step or multiple-step process. The core particles 102 are synthesized first and the shell 104 is then formed on the core particle 102 via different methods, depending on the type of core and shell materials and their morphologies. The drive in the preparation of core-shell particles is to combine the desired properties of different materials and structures in order to offer synergistic effect, to stabilize the active particles, or to provide biocompatible properties. Therefore, anti-bacterial particles need treatment process for the core 102 and the shell 104 surface, activated by chemical reagents.

The method for preparing the long-lasting antibacterial core-shell agent 100, comprises the steps of: synthesizing an anti-bacterial core having an antibacterial metal powder, a poorly soluble metal salt and a metal oxide as indicated at block 202. The synthesis of the anti-bacterial core comprises the steps of: preparing a surfactant solution of a cationic surfactant and an organic solvent with a concentration of 0.01-0.5 M as indicated at block 204. Then adding a certain amount of aqueous metal nitrate solution with a concentration of 0.05-0.1 M to the surfactant solution by fixing the mole ratio of water to surfactant as 4 to 6, to form a water/oil micro-emulsion as indicated at block 206. Stirring the water/oil micro-emulsion at a constant speed at room temperature and then adding a reducing agent drop-wise to reduce metal ions to metal as indicated at block 208. The reaction mixture changed from highly transparent to dark color due to the formation of metal colloidal system. Then preparing nano particles of metal oxide by adjusting the hydrolysis ratio and adding a protective agent as indicated at block 210 and preparing the poorly soluble metal salt from at least two soluble metal salts by mechanochemical processing and precipitation reaction synthesis as indicated at block 212. A porous oxide shell is formed over the anti-bacterial core by fluidizing and agitating an organic polymer as indicated at block 214.

Thus for making the antibacterial metal powder, a solution of cationic surfactant in organic solvent with a concentration of 0.01-0.5 M was prepared. In this embodiment, permanently charged quaternary ammonium cations are used as surfactants which include alkyltrimethylammonium salts like cetyl trimethylammonium bromide (CTAB) and cetyl trimethylammonium chloride (CTAC). The organic solvent includes alcohols selected from the group consisting of: ethanol and isopropyl alcohol.

Then by fixing the mole ratio of water to surfactant as 4 to 6, a certain amount of aqueous metal nitrate solution with a concentration of 0.05-0.1 M was added to the surfactant solution to form a water/oil microemulsion system. The mixture was stirred at a constant speed at room temperature and then the reducing agent was added dropwise to reduce metal ions to metal. The reducing agent used in this embodiment includes antioxidant molecules selected from the group consisting of: sodium borohydride and ascorbic acid. The reaction mixture changed from highly transparent to dark color due to the formation of metal colloidal system. The antibacterial metal powder is made from metals selected from the group consisting of: Mercury (Hg), Silver (Ag), Arsenic (As), Copper (Cu), Zinc (Zn), Cobalt (Co), Nickel (Ni), Aluminum (Al), Titanium (Ti) and Manganese (Mn).

Wet-chemical methods including hydrothermal/solvothermal processes, solution-liquid-solid, and surfactant-assisted synthesis were employed for making the metal oxide. These methods provide a convenient and facile platform for a low-temperature fabrication of the metal oxide nanostructures. Typical precursors for metal oxide preparation are metal nitrate, metal chloride, metal acetate, and metal sulfate. If an anisotropic growth of metal oxide nanoparticles is desired, surfactants such as hexamethylenetetramine, ammonia, ascorbic acid, and sodium hydroxide are added. Most reactions are performed at elevated temperatures up to 180° C. Different kinds of oxide nanoparticles are obtained by adjusting the hydrolysis ratio. An important parameter for metal based particle is the hydrolysis ratio, defined as h=nH₂O/nmetal (mol/mol). Varying h allows to prepare either metal, (oxy)hydroxide or oxide nanoparticles. The example for Al is Al particle, Al(OH)₃, and Al₂O₃.

The nature of the protective agent added during oxide formation and hydrolysis ratio are two major handles for size and shape control. The protective agents used for preparing metal oxides are surfactants and polymers. In this embodiment, dimethylaminoborane and poly(acrylic acid, sodium salt) is used.

The commercial production of salt nanoparticles is realized by mechanochemical processing and precipitation reaction synthesis. The first method is based on physical size reduction in a conventional ball mill with additives that are activated during grinding. The additives are polymers and in this embodiment, poly(acrylic acid, sodium salt) is used. The reaction comprises the mechanical activation of precursors which includes at least two kinds of soluble metal salts, with a further thermal decomposition to poorly soluble metal salts. The at least two kinds of soluble metal salts are nitrates of silver, copper or mercury mixed with sodium chloride to form AgCl, CuCl, HgCl₂. In this embodiment, the three nitrates can be used alone or combinations of any two or three nitrates can be used. The particle size can be varied by milling time and the heat treatment temperature. The poorly soluble metal salt is selected as from the group consisting of: Silver Chloride (AgCl), Copper Chloride (CuCl) and Mercury Chloride (HgCl₂).

In the present embodiment, the porous oxide shell 104 over the anti-bacterial core 102 is formed by fluidizing and agitating an organic polymer. The particles are fluidized or agitated to perform the layer surface reactions in reasonable times and to prevent the particles from being aggregated by the polymer film. A variety of pure organic polymers as well as hybrid organic-inorganic polymer films, such as alucone, silicon alkoxide (silicone), zinc alkoxide (zincone), titanium alkoxide (titanicone), and zirconium alkoxide (zircone) can be utilized for the growth of the porous oxide shell 104. As indicated at block 214, the fluidizing and agitating of the organic polymer is done to perform the layer surface reactions in reasonable times and to prevent the particles from being aggregated by the polymer film.

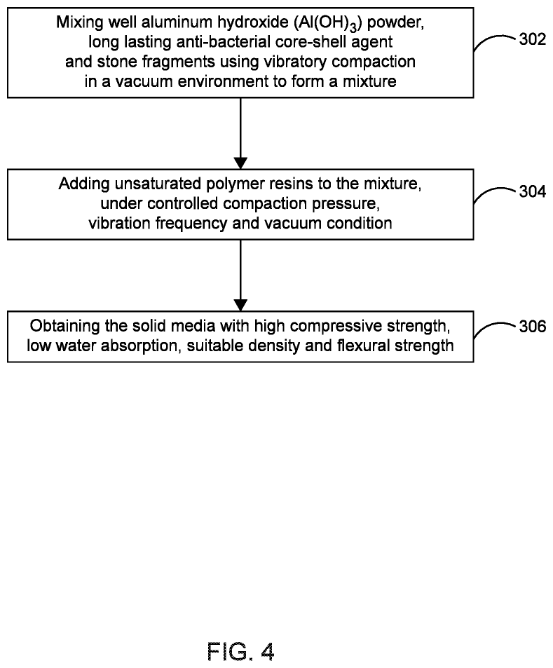

FIG. 4 illustrates a method for preparing a solid media utilizing the long-lasting antibacterial core-shell agent 100 in accordance with the preferred embodiment of the present invention. The method comprises the steps of: mixing well aluminum hydroxide (Al(OH)₃) powder, the long lasting anti-bacterial core-shell agent and stone fragments using vibratory compaction in a vacuum environment to form a mixture as indicated at block 302. Adding unsaturated polymer resins to the mixture, under controlled compaction pressure, vibration frequency and vacuum condition as indicated at block 304 and obtaining the solid media with high compressive strength, low water absorption, suitable density and flexural strength as indicated at block 306.

Thus, in the present embodiment, aluminum hydroxide (Al(OH)₃) powder, the long lasting anti-bacterial core-shell agent 100, and stone fragments from stone slab processing are used as raw materials for making artificial stone slabs using vibratory compaction in a vacuum environment. All powders are well mixed firstly before use. The percentage of each component of the solid media includes 60-69% of Al(OH)₃ powder, 1-10% of the long lasting anti-bacterial core-shell agent, 10% of stone fragments and 8% of unsaturated polymer resins. Under controlled compaction pressure, vibration frequency and vacuum condition, artificial stone slabs with high compressive strength, low water absorption, suitable density, and flexural strength are obtained after minutes compaction. The stone fragments includes fine granite aggregates. The method of the present embodiment creates artificial stone slabs of superior quality in terms of strength compared as to natural construction slabs and exhibits well dispersion of anti-bacterial characteristics.

The solid media thus produced from the present method utilizing the long-lasting antibacterial core-shell agent 100 have the ability to resist ultraviolet damage, inhibit oxygen oxidation, and slowly release metal ions. The solid media has long-term release of antibacterial ions and has longitudinal antibacterial uniformity. The solid media can maintain a consistent antibacterial ability after various surface grinding and treating processes and the unique ratio of the unsaturated polymer resins resin allows the antibacterial component to be released continuously. The antibacterial characteristic of the solid media is consistent from 0.1 mm to 12 mm.

In order to improve the long lasting ability, one-pot synthesis producing dense silica coated shells with periodic mesoporous organosilica. The core 102 has a passive function in long-term metal ion releasing, as the impermeability of dense silica was intended to extend diffusion paths for long lasting anti-bacterial applications.

Mesoporous silicas are often used as the outer shell 104 in the construction of core-shell nanoparticles. Then, the large permanent cargo space can be used to store and release substances, and reactive silanol groups on the surface enable modifications via post-grafting methods and therefore allow for an adaptation of the surface chemistry to the intended application.

The periodic mesoporous organosilicas offer similar advantages as silica. They are built up from bridged bis-silylated organosilanes of the type (RO)₃Si—R′—Si(OR)₃ providing the inherent and homogeneously distributed organic functionalities within the highly porous network. They are also assumed to be biocompatible for bacterial cells and offer the options for further modifications via post-grafting or via functionalization of the organic bridging units.

In this embodiment, the construction of core-shell nanoparticles with a controllable shell of mesoporous silica or phenylene-bridged organosilica is used. The different hydrophilicities of two shell materials (hydrophilic silica/hydrophobic organosilica) are employed for the autonomous selective adsorption of acrylic resin bonding agents at different cases of artificial stone products.

Also, based on the sample principle, the formation of porous SiO₂ can be replaced by Al₂O₃, TiO₂, ZrO₂ and ZnO with the thickness range of 10 to 1500 nm.

The foregoing description of the preferred embodiment of the present as invention has been presented for the purpose of illustration and description. It is not intended to be exhaustive or to limit the invention to the precise form disclosed. Many modifications and variations are possible in light of the above teachings. It is intended that the scope of the present invention not be limited by this detailed description, but by the claims and the equivalents to the claims appended hereto.

What is claimed is:

1. A long-lasting antibacterial core-shell agent, comprising:
an antibacterial core composed of an antibacterial metal powder, a poorly soluble metal salt and a metal oxide; and
an outer shell composed of a porous oxide;
whereby the structure has the ability to resist ultraviolet damage, inhibits oxygen oxidation, slowly releases metal ions and thereby exhibits long-term release of antibacterial ions and longitudinal antibacterial uniformity.

2. The long-lasting antibacterial core-shell agent of claim 1 wherein the antibacterial metal powder is nano-sized.

3. The long-lasting antibacterial core-shell agent of claim 1 wherein the antibacterial metal powder is made from metals selected from the group consisting of: Mercury (Hg), Silver (Ag), Arsenic (As), Copper (Cu), Zinc (Zn), Cobalt (Co), Nickel (Ni), Aluminum (Al), Titanium (Ti) and Manganese (Mn).

4. The long-lasting antibacterial core-shell agent of claim 1 wherein the size of the particles of the antibacterial metal powder can range from 30 nm to 3000 nm.

5. The long-lasting antibacterial core-shell agent of claim 1 wherein the poorly soluble metal salt is selected from the group consisting of Silver Chloride (AgCl), Copper Chloride (CuCl) and Mercury Chloride (HgCl₂).

6. The long-lasting antibacterial core-shell agent of claim 1 wherein the metal oxide is selected from the group consisting of: Copper Oxide (CuO) and Zinc Oxide (ZnO).

7. The long-lasting antibacterial core-shell agent of claim 1 wherein metal oxides of metals selected from the group consisting of: Mercury (Hg), Silver (Ag), Arsenic (As), as Cobalt (Co), Nickel (Ni), Aluminum (Al), Titanium (Ti) and Manganese (Mn) can also be employed.

8. The long-lasting antibacterial core-shell agent of claim 1 wherein the porous oxide is selected from the group consisting of: silicon dioxide (SiO₂), titanium dioxide (TiO₂), aluminum oxide (Al₂O₃) and aluminum hydroxide (Al₂(OH)₃).

9. The long-lasting antibacterial core-shell agent of claim 1 wherein the thickness of the particles of porous oxide can range from 10 nm to 1500 nm.

10. The long-lasting antibacterial core-shell agent of claim 1 wherein the antibacterial core acts as slow-releasing centers of metal ions.

11. A method for preparing a long-lasting antibacterial agent, the method comprising the steps of:
a) synthesizing an anti-bacterial core having an antibacterial metal powder, a poorly soluble metal salt and a metal oxide, the synthesis comprising the steps of:
i. preparing a surfactant solution of a cationic surfactant and an organic solvent with a concentration of 0.01-0.5 M;
ii. adding a certain amount of aqueous metal nitrate solution with a concentration of 0.05-0.1 M to the surfactant solution by fixing the mole ratio of water to surfactant as 4 to 6, to form a water/oil micro-emulsion;
iii. stirring the water/oil micro-emulsion at a constant speed at room temperature and then adding a reducing agent drop-wise to reduce metal ions to metal;
iv. preparing nano particles of metal oxide by adjusting the hydrolysis ratio and adding a protective agent; and
v. preparing poorly soluble metal salt from at least two soluble metal salts by mechanochemical processing and precipitation reaction synthesis; and
b) forming a porous oxide shell over the anti-bacterial core by fluidizing and agitating an organic polymer.

12. The method of claim 11 wherein the antibacterial metal powder is made from metals selected from the group consisting of: Mercury (Hg), Silver (Ag), Arsenic (As), Copper (Cu), Zinc (Zn), Cobalt (Co), Nickel (Ni), Aluminum (Al), Titanium (Ti) and Manganese (Mn).

13. The method of claim 11 wherein the poorly soluble metal salt is selected from the group consisting of: Silver Chloride (AgCl), Copper Chloride (CuCl) and Mercury Chloride (HgCl₂).

14. The method of claim 11 wherein the metal oxide is selected from the group consisting of copper oxide (CuO) and zinc oxide (ZnO).

15. The method of claim 11 wherein the metal oxide is made from precursors selected from the group consisting of: metal nitrate, metal chloride, metal acetate and metal sulfate.

16. The method of claim 11 wherein the organic polymer for making the porous oxide is selected from the group consisting of: alucone, silicon alkoxide (silicone), zinc alkoxide (zincone), titanium alkoxide (titanicone), and zirconium alkoxide (zircone).

17. The method of claim 11 wherein the fluidizing and agitating of the organic polymer is done to perform the layer surface reactions in reasonable times and to prevent the particles from being aggregated by the polymer film.

18. The method of claim 11 wherein the porous oxide is selected from the group consisting of silicon dioxide (SiO₂), titanium dioxide (TiO₂), aluminum oxide (Al₂O₃) and aluminum hydroxide (Al₂(OH)₃).

19. A method for preparing a solid media utilizing a long-lasting antibacterial core-shell agent, the method comprising the steps of:
a) mixing well aluminum hydroxide (Al(OH)₃) powder, long lasting anti-bacterial core-shell agent and stone fragments using vibratory compaction in a vacuum environment to form a mixture;
is b) adding unsaturated polymer resins (8%) to the mixture, under controlled compaction pressure, vibration frequency and vacuum condition; and
c) obtaining the solid media with high compressive strength, low water absorption, suitable density and flexural strength.

20. The method of claim 19 wherein the percentage of each component of the solid media includes 60-69% of Al(OH)3 powder, 1-10% of the long lasting anti-bacterial core-shell agent, 10% of stone fragments and 8% of unsaturated polymer resins.

21. The method of claim 19 wherein the stone fragments includes fine granite aggregates.

22. The method of claim 19 creates artificial stone slabs of superior quality in terms of strength compared to natural construction slabs and exhibits well dispersion of anti-bacterial characteristics.

23. The method of claim 19 wherein the solid media have the ability to resist ultraviolet damage, inhibit oxygen oxidation, and slowly release metal ions.

24. The method of claim 19 wherein the solid media has long-term release of antibacterial ions and has longitudinal antibacterial uniformity.

25. The method of claim 19 wherein the solid media can maintain a consistent antibacterial ability after various surface grinding and treating processes and the unique ratio of the unsaturated polymer resins resin allows the antibacterial component to be released continuously.

26. The method of claim 19 wherein the antibacterial characteristic of the solid media is consistent from 0.1 mm to 12 mm.
